(19) United States
(12) Patent Application Publication
(10) Pub. No.: US 2005/0049692 A1
(43) Pub. Date: Mar. 3, 2005

(54) Medical device for reduction of pressure effects of cardiac tricuspid valve regurgitation

(76) Inventors: Michael J. Numamoto, Irvine, CA (US); Rodolfo C. Quijano, Laguna Hills, CA (US); Hosheng Tu, Newport Beach, CA (US)

Correspondence Address: Rodolfo C. Quijano, Suite 100, 2 Jenner, Irvine, CA, 92618, US

(21) Appl. No.: 10/653,397

(22) Filed: Sep. 2, 2003

(51) Int. Cl. A61F 2/06; A61F 2/24

(52) U.S. Cl. 623/1.24; 623/2.14

(57) ABSTRACT

An elongate valve stent and methods for protecting an upper or a lower body of a patient from high venous pressures comprising a stent member, the stent member comprising a support structure and a tissue valve, wherein the tissue valve is configured to permit fluid flow in one direction and prevent fluid flow in an opposite direction, and means for anchoring the stent member onto surrounding tissue of the superior vena cava or inferior vena cava.

### FIELD OF THE INVENTION

The present invention relates generally to stented venous valves and, more particularly, to stented valve bioprostheses with fixation means and methods for reduction of pressure effects of cardiac tricuspid valve regurgitation.

### BACKGROUND OF THE INVENTION

Among the quadruped heart valves in a human body, the tricuspid valve separates the right atrium (upper chamber) from the right ventricle (lower chamber), and channels the venous blood return to the heart on its way to the lungs. When the venous blood is impelled to the lung arteries, this tricuspid valve closes to block the blood return from backflowing to the atrium and thus provides efficiency to the ejection of blood from the right ventricle that directs the flow towards the lung. In instances where the tricuspid valve is unable to close properly, the pumping pressure of the ventricle can be transmitted in reverse to the atrium and subsequently to the vena cavae. Typically, the superior vena cava functions to bring blood to the heart from the head and the inferior vena cava functions to bring blood to the heart from the liver and other parts of the body (kidneys, gut, legs) that are located below the heart. This pressure can have deleterious effects on the work of the heart and circulatory system. The device herein described provides means of reduction or total nullification of the effects of pressure on the channels of venous return to the heart.

The tricuspid heart valve has an area close to 10 square centimeters, and a circumference approaching 12 centimeters. As the name implies it has three cusps or leaflets that separate to open the valve and allow the venous return from the body to the heart to enter the pumping chamber or right ventricle that redirects the flow towards the lung where venous blood is oxygenated and transformed into arterial blood to supply all tissues of the body. During the pumping action, the tricuspid valve closes to impede retrograde flow into the right atrium.

Acquired disease of the tricuspid valve is much less common than that of the other valves of the heart; this is a reflection of the lower pressures that are experienced by the right chambers of the heart, and thus, the valves of the right side of the heart function generally under less stresses than its left side counterparts. Disease can affect the tricuspid valve mostly in two forms, 1) as tricuspid valve stenosis, a restriction of the opening of the valve, most likely of rheumatic origin, and 2) as tricuspid valve regurgitation or incompetence, generally due to any disease process that causes alterations in the tricuspid valve apparatus that consists of: leaflets, chords, tendinous material that join the leaflet to the muscle of the right side of the heart, or the annulus (the ring of tissue where the leaflets join the atrium). In the latter, the valve is unable to close completely thus allowing retrograde flow or regurgitation from the ventricle into the atrium.

A small degree of tricuspid regurgitation is found in normal hearts and the prevalence increases with age. Physiologically, the regurgitation is seen as a jet whose velocity is proportional to the pressure differential between the right ventricle and the right atrium. Tricuspid regurgitation (TR) alone may be well tolerated. However, patients suffering from severe TR are troubled with swelling of the legs, pulsations of the jugular vein pulse at the neck due to reverse flow and pressure into the superior vena cava. Other problems associated with severe TR include liver congestion due to reverse pressure to the inferior vena cava and the liver veins, and fatigue and general malaise because of decreased pumping of blood through the heart (that is, decreased cardiac output), that may progress to cardiac cirrhosis and liver dysfunction with prolonged hepatic congestion. Furthermore, high venous pressure may contribute to renal dysfunction and other symptoms of abdominal bloating. All these findings are dependent on the severity of tricuspid regurgitation and pulmonary hypertension. Often the end effect is right heart failure.

Tricuspid regurgitation can be alleviated or eliminated by surgical means, either by replacement of the total valve apparatus with an artificially fabricated replacement tricuspid heart valve, or by constriction of the valve ring with means of an annular remodeling ring (annuloplasty ring). The tricuspid valve repair is not always 100% effective in eliminating the TR, as it has been found in some instances that patients (up to about 15%) who have undergone tricuspid valve annuloplasty may leave the hospital with moderate to severe TR and the tricuspid dysfunction rate may steadily increase to about 30-50%. If surgery is impossible to perform, i.e., if the patient is deemed inoperable or operable only at a too high surgical risk, an alternative possibility is to treat the patient with a stented valvular device and percutaneous means of device delivery for protecting the upper and/or lower body from high venous pressures.

U.S. Pat. No. 6,503,272 issued on Jan. 7, 2003, entire contents of which are incorporated herein by reference, discloses an artificial venous valve which incorporates a stent having one or more of the elements comprising its frame deformed inwardly towards its center and a biocompatible fabric attached to the one or more elements utilized to replace or supplement incompetent or damaged venous valves.

U.S. Pat. No. 5,855,601 issued on Jan. 5, 1999, entire contents of which are incorporated herein by reference, discloses an artificial venous valve comprising a tubular valve segment containing venous valve means and at least one self-expanding, cylindrical stent member having a plurality of barbs extending from the outer surface of the stent member to engage the natural tissue of the site to hold the valve in place after implantation.

U.S. Pat. No. 6,299,637 issued on Oct. 9, 2001, entire contents of which are incorporated herein by reference, discloses a self expandable prosthetic venous valve comprising a tubular wire support, expandable from a first reduced diameter to a second enlarged diameter, and at least one leaflet pivotably positioned in the flow path for permitting flow in a forward direction and resisting flow in a reverse direction.

U.S. Pat. No. 5,824,061 issued on Oct. 20, 1998, entire contents of which are incorporated herein by reference, discloses an endovascular venous valve prosthesis comprising an endovascular stent assembly including a stent having a generally cylindrical body with a hollow bore extending longitudinally therethrough and first and second support struts formed on opposite sides of the outflow end of the cylindrical body and extending generally longitudinally therefrom; and a preserved segment of vein having an outer wall and a venous valve positioned therein, the valve having two leaflets extending generally longitudinally within the segment of vein with lateral edges adjacent the outer wall.

U.S. Pat. No. 5,607,465 issued on Mar. 4, 1997, entire contents of which are incorporated herein by reference, discloses a valve for use in a blood vessel having a bent flexible wire mesh with elasticity and plasticity so as to be collapsible and implantable remotely at a desired site and a monocusp sail-like valving element mounted onto it.

U.S. Pat. No. 5,997,573 issued on Dec. 7, 1999, entire contents of which are incorporated herein by reference, discloses a dilation restrictor apparatus for limiting the extent to which a blood vessel may dilate adjacent to a point whereat a cut end of the blood vessel has been anastomosed to a venous valve implant, the dilation restrictor apparatus comprising an elongate tubular body having a hollow bore containing a plurality of apertures formed therein to permit passage of fluid therethrough.

U.S. Pat. No. 6,383,193 issued on May 7, 2002, entire contents of which are incorporated herein by reference, discloses a delivery system for the percutaneous insertion of a self-expanding vena cava filter device being formed with a length along a longitudinal filter axis, the system comprising constraining the filter in a compact condition within an elongated, radially flexible and axially stiff tubular member and a displacement member attached to the tubular member for displacing the filter from the segment thereby to deploy the filter.

None of the above-referenced prior art discloses means for protecting the upper body and/or lower body of a patient from spiked or elevated venous pressure resulting from cardiac tricuspid valve regurgitation.

Co-pending patent application Ser. No. 10/418,677, filed on Apr. 17, 2003, entire contents of which are incorporated herein by reference, discloses an elongate valve stent comprising a first end, a middle section, and an opposite second end that is connected to the first end with at least one elongate connecting member, a first stent member disposed at and secured to the first end, the first stent member comprising a first support structure and a first tissue valve, and a second stent member disposed at and secured to the second end, the second stent member comprising a second support structure and a second tissue valve.

Another co-pending patent application Ser. No. 10/418,663, filed on Apr. 17, 2003, entire contents of which are incorporated herein by reference, discloses a method of protecting an upper body and a lower body of a patient from high venous pressures comprising implanting a first valve at a superior vena cava and a second valve at an inferior vena cava, wherein the first and second valves are configured to permit blood flow towards a right atrium of the patient and prevent blood flow in an opposite direction. However, means for anchoring the device has not been fully disclosed.

Therefore, it is one preferred object to provide a method of protecting an upper body and/or a lower body of a patient from high venous pressures comprising implanting an elongate valve stent having a valved stent member placed at a superior vena cava and/or at an inferior vena cava, wherein the stent member is equipped with anchoring means for securely anchoring the device at an appropriate vena cava location. It is another preferred object to provide a valve stent device with a venous filtering capability.

### SUMMARY OF THE INVENTION

In general, it is one object of the present invention to provide a stented valve bioprosthesis and methods for reduction of pressure effects of cardiac tricuspid valve regurgitation.

In one aspect of the invention, it is provided an elongate valve stent comprising a stent member, the stent member comprising a support structure that is collapsible and expandable and a tissue valve, wherein the tissue valve is configured to permit fluid flow in one direction and prevent fluid flow in an opposite direction and means for anchoring the stent member onto surrounding tissue of a blood vessel.

In another aspect of the invention, it is provided an elongate valve stent comprising a stent member, the stent member comprising a support structure and a tissue valve, wherein the tissue valve is configured to permit fluid flow in one direction and prevent fluid flow in an opposite direction, and means for filtering the fluid of a blood vessel. In one embodiment, the blood vessel is a vein, a superior vena cava or an inferior vena cava. In another embodiment, a filter member is mounted at an upstream side of the stent member.

In some aspect of the invention, it is provided a method of protecting an upper or a lower body of a patient from high venous pressures comprising: providing an elongate valve stent, wherein the stent comprises a stent member with a tissue valve secured to a support structure, wherein the support structure is collapsibly expandable, and anchoring means for anchoring the stent member onto surrounding tissue of a vena cava; passing the elongate valve stent through a blood vessel with the support structure in a collapsed position; deploying the stent to an inferior vena cava or a superior vena cava with the support structure in an expanded shape; and securing the stent by anchoring the stent member onto the surrounding tissue of either the superior vena cava or the inferior vena cava with the anchoring means.

In a preferred aspect of the invention, at least a portion of the elongate valve stent is coated with a therapeutic agent, wherein the therapeutic agent is selected from a group consisting of anticoagulants, antithrombogenic agents, anti-proliferative agents, anti-inflammatory agents, antibiotics, stem cells, growth factors, angiogenesis agents, anti-angiogenesis agents, and statins.

### BRIEF DESCRIPTION OF THE DRAWINGS

Additional objects and features of the present invention will become more apparent and the invention itself will be best understood from the following Detailed Description of Exemplary Embodiments, when read with reference to the accompanying drawings.

FIG. 1 is a front view of a stent member of an elongate valve stent according to the principles of the present invention.

FIG. 2 is a side view of the stented valve of FIG. 1.

FIG. 3 is a cross-sectional view of the stent strut, section I-I, of the stented valve in FIG. 1.

FIG. 4 is a preferred embodiment of an elongate valve stent with anchoring means in accordance with the principles of the present invention.

FIG. 5 is another preferred embodiment of an elongate valve stent with filtering means in accordance with the principles of the present invention.

FIG. 6 shows a delivery apparatus with an elongate valve stent at a collapsed position during a delivery phase.

FIG. 7 shows a delivery apparatus with an elongate valve stent at a partially expanded position during a positioning phase.

FIG. 8 is an illustrated procedure of implanting an elongate valve stent having anchoring means, wherein a stent member with a tissue valve is placed at the inferior vena cava configured to permit blood flow towards the right atrium of a patient.

### DETAILED DESCRIPTION OF EXEMPLARY EMBODIMENTS

The preferred embodiments of the present invention described below relate particularly to venous valve bioprostheses and methods for reduction of pressure effects of cardiac tricuspid valve regurgitation. While the description sets forth various embodiment specific details, it will be appreciated that the description is illustrative only and should not be construed in any way as limiting the invention. Furthermore, various applications of the invention, and modifications thereto, which may occur to those who are skilled in the art, are also encompassed by the general concepts described below.

A stented valve or valve stent is a device to be placed inside a channel of the body that allows fluid flow in one direction and prevents fluid flow in an opposite direction. In a normal person, the superior vena cava functions to bring blood to the heart from the head and the inferior vena cava functions to bring blood to the heart from the liver and other parts of the body (kidneys, gut, legs) that are located below the heart.

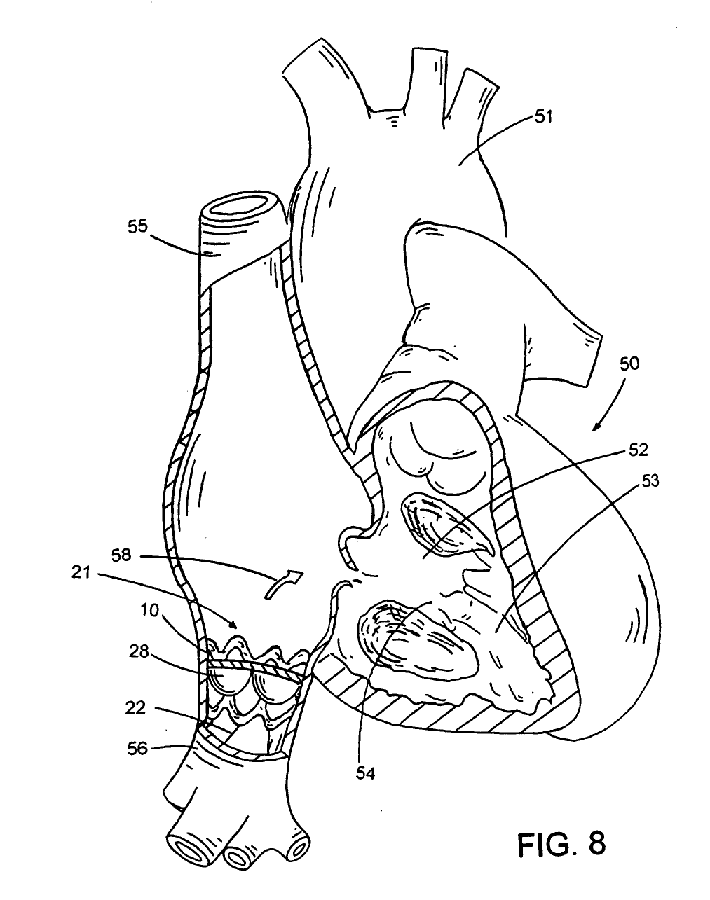

In instances where the tricuspid valve (54 in FIG. 8) is unable to close properly, the pumping pressure of the ventricle 53 can be transmitted in reverse to the atrium 52 and subsequently to the vena cavae 55, 56. This pressure can have deleterious effects on the work of the heart and circulatory system. It is one aspect of the invention to provide a device and methods enabling reduction or total nullification of the effects of elevated pressure on the channels of venous return to the heart.

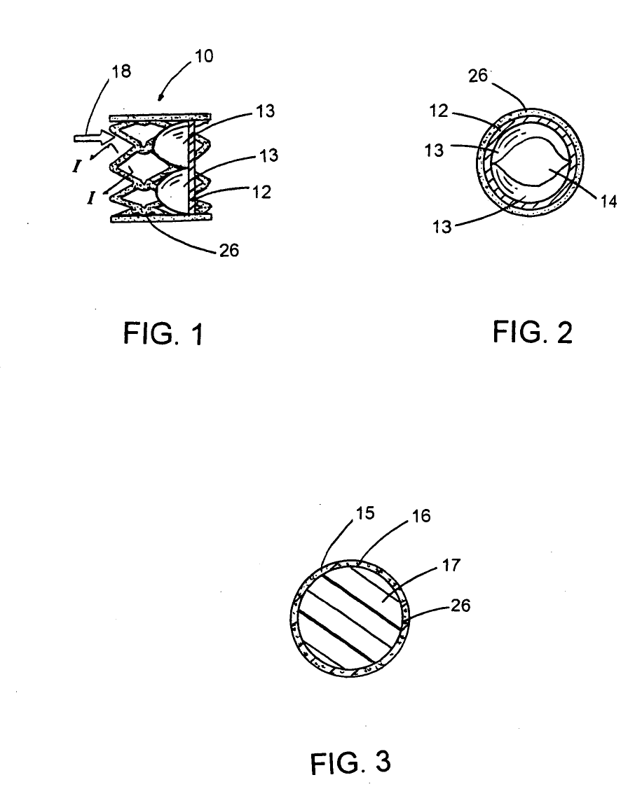

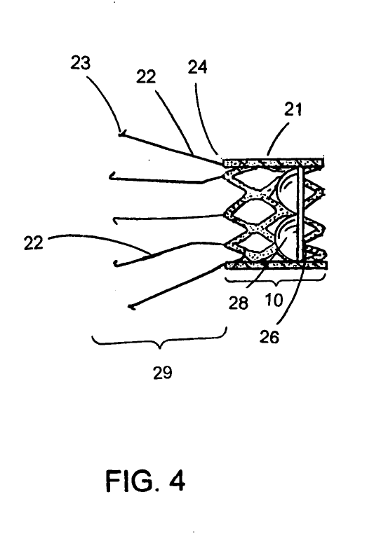

FIG. 1 shows a front view of a stent member 10 of an elongate valve stent while FIG. 2 shows its side view according to the principles of the present invention. Some aspects of the invention relate to an elongate valve stent (21 in FIG. 4) comprising a stent member 10, the stent member comprising a support structure 26 and a tissue valve 28, wherein the tissue valve is configured to permit fluid flow in one direction and prevent fluid flow in an opposite direction, and means 29 for anchoring the stent member onto surrounding tissue of a blood vessel, such as a vein or a vena cava.

The stent member 10 comprises a tissue valve that is secured to a support structure 26, wherein the support structure is collapsibly expandable (that is, collapsible and expandable). The tissue valve comprises at least one leaflet 13 securely attached to an annular base 12. The tissue valve is configured to permit fluid flow in a first direction (as shown by the arrow 18) and prevent fluid flow in an opposite direction. When the fluid flows in the first direction, the leaflet 13 is open having a flow-through opening 14.

In one embodiment, the support structure 26 of the stent member 10 is self-expandable out of a delivery apparatus 31. In one embodiment of operations, the stent is compressed radially to be held within the lumen of the delivery apparatus, sheath, catheter, applicator, or cannula. Upon delivery out of the apparatus 31, the stent self-expands to its pre-compressed state. The stent is typically made of a material selected from a group consisting of stainless steel, Nitinol, plastics or the like, particularly the shape-member material with flexibility and strength. In another embodiment, the stent member 10 of the valve stent 21 is expandable by an inflatable balloon, which is well known to an ordinary artisan who is skilled in the art.

In still another embodiment, the support structure 26 is made of a shape-memory material having a first shape transition temperature of between about 30° C. and 45° C. and a second shape transition temperature of between about 25° C. and −20° C., preferably between about 5° C. and −10° C. In operations, the stent is collapsibly deformed to a small diameter and held at about or below 5° C., preferably between about 5° C. and −10° C. The deformed stent is then inserted within a delivery apparatus 31. During a delivery phase, the stent is maintained at below the second shape transition temperature by flushing or contacting with super-cooled saline. At a desired location, the stent is pushed out of the sheath of the delivery apparatus. Upon reaching the first shape transition temperature, the stent expands to lock itself in position.

The use of shape memory alloys or intermetallics and, specifically, Nitinol in the construction of medical devices is well known. U.S. Pat. No. 6,451,025 issued on Sep. 17, 2002, entire contents of which are incorporated herein by reference, discloses hysteresis behavior of Nitinol to generate shape change or force at or around constant body temperature by forming the device to the final shape desired, straining the device in a direction which tends to facilitate placement into the body, restraining the device in this strained shape during insertion into or placement near the body, then releasing all or part of the device such that it returns or tends to return to the desired shape with temperature activation.

In one aspect, the first valve stent 21 is delivered to the superior vena cava 55 endoluminally from a subclavian or femoral vein. In another aspect, the second valve stent is delivered from a femoral vein or jugular vein to the inferior vena cava 56.

The step of delivering the elongate valve stent endoluminally is through an incision at a blood vessel selected from a group consisting of a jugular vein, a femoral vein, a subclavian vein or other veins. The stent member is expanded from a collapsible position when the stent member reaches an appropriate site. In a further aspect, the valve stent 21 further comprises anchoring means 29 for anchoring the stent onto surrounding tissue of either the superior vena cava or the inferior vena cava, for example, hooks, barbs, needles, protrusion, or the like. By way of example, U.S. Pat. No. 6,610,085, entire contents of which are incorporated herein by reference, discloses anchoring means that is well known to one who is skilled in the art.

In an alternate embodiment, the venous valve to be placed at either the superior vena cava or the inferior vena cava is a stentless valve. In still another embodiment, the venous valves are to be implanted by an open chest procedure at the superior vena cava and/or the inferior vena cava, wherein the valves can be either a stented valve or a stentless valve.

In a preferred embodiment, the valve stent 21 would deploy in the superior vena cava 55 just above the right atrial junction but below the azygos vein. Alternately, the valve stent would deploy in the inferior vena cava 56 just below the right atrium 52 but above the hepatic veins. In effect, the physiologic changes from the therapy disclosed herein would be to protect the upper and/or lower body from high or elevated venous pressures. Patients with severe tricuspid regurgitation are troubled by ascites, peripheral edema frequently with stasis changes in the legs, hepatic congestion, which may progress to cardiac cirrhosis and liver dysfunction with prolonged hepatic congestion. Furthermore, high venous pressure may contribute to renal dysfunction and other symptoms of abdominal bloating. The neck vein and upper body congestion is sometimes quite visible in patients including the pulsatile neck veins. By placing the valve stents of the invention, it should protect the patient from ascites, hepatic congestion, edema and the eventual development of cardiac cirrhosis.

To enhance the biocompatibility of the device or improved therapy to the surrounding tissue, it is provided that at least a portion of the stent member 10 of the elongate valve stent 21 is coated with a therapeutic agent, wherein the therapeutic agent is selected from a group consisting of anticoagulants, antithrombogenic agents, anti-proliferative agents, anti-inflammatory agents, antibiotics, stem cells, growth factors, angiogenesis agents, anti-angiogenesis agents, and statins. The therapeutic agent is to slowly release to the tissue or blood stream at an effective amount over time.

For illustration purposes, FIG. 3 shows a cross-sectional view of the stent strut 17 of the support structure 26, section I-I, of the stent member 10 in FIG. 1, wherein a polymer layer 16 is coated onto the periphery surface of the stent strut 17 and the polymer layer 16 is loaded with the desired therapeutic agent 15 for slow release at an effective amount over time to the surrounding tissue.

Many medical materials used in the treatment of cardiovascular diseases are required to possess biocompatible and hemo-compatible properties with reduced antigenicity. One method to treat tissue so as to render the tissue more suitable as a biomaterial is a process called chemical treatment. Several chemical treatment agent and methods have been disclosed. Among them, aldehydes (glutaraldehyde, formaldehyde, dialdehyde starch and the like), epoxy compounds, genipin, and their analog or derivatives thereof are all applicable in treating a tissue. Chemical treatment conditions and procedures to render the tissue suitable as a biomaterial depend on the property of each tissue and intended medical applications, wherein the conditions/procedures are well documented in published literature and well known to one who is skilled in the art.

The tissue valve 28 of the stent member 10 has at least one valve leaflet 13. Sometimes, the tissue valve may have two, three or more leaflets. In some aspect of the present invention, the leaflet 13 is made from a pericardium, the pericardium being selected from a group consisting of a bovine pericardium, an equine pericardium, a porcine pericardium, an ovine pericardium and the like. Further, the tissue valve is chemically treated with a chemical treating agent selected from a group consisting of glutaraldehyde, formaldehyde, dialdehyde starch, epoxy compounds, genipin, and mixture thereof. In one embodiment, the tissue valve is a venous valve selected or procured from a group consisting of a bovine jugular vein, an equine jugular vein, a porcine jugular vein, and an ovine jugular vein. In another embodiment, the tissue valve is a porcine valve.

U.S. Pat. No. 4,806,595 issued on Feb. 21, 1989, entire contents of which are incorporated herein by reference, discloses a novel method for preparing medical materials by using epoxy compounds as chemical treatment agent for tissue, wherein the “epoxy compounds” include glycol diglycidyl ether, polyol polyglycidyl ether, dicarboxylic acid diglycidylester, the analog, and derivatives thereof.

U.S. Pat. No. 6,608,040 issued on Aug. 19, 2003, entire contents of which are incorporated herein by reference, discloses a novel method for preparing medical materials by using genipin as chemical treatment agent for tissue.

FIG. 4 shows a preferred embodiment of an elongate valve stent with anchoring means 29 in accordance with the principles of the present invention. In some aspect, it is provided an elongate valve stent 21 comprising a stent member 10, the stent member comprising a support structure 26 and a tissue valve 28, wherein the tissue valve is configured to permit fluid flow in one direction and prevent fluid flow in an opposite direction. The anchoring means 29 for anchoring the stent member 10 onto surrounding tissue of a blood vessel comprises at least one anchoring member 22, wherein each anchoring member 21 comprises a proximal end 24 connected to one end of the stent member 10 and a distal end with a needle or hook 23 for penetrating and hooking into tissue. In one preferred embodiment, the tissue valve 28 has at least one valve leaflet 13 sized and configured to permit fluid flow in one direction (shown by an arrow 58) and prevent fluid flow in an opposite direction.

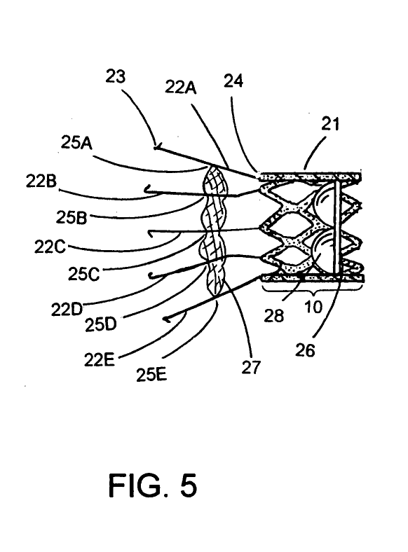

FIG. 5 shows another preferred embodiment of an elongate valve stent 21 with filtering means 27 in accordance with the principles of the present invention. Some aspects of the invention relate to an elongate valve stent 21 comprising a stent member 10, the stent member comprising a support structure 26 and a tissue valve 28, wherein the tissue valve is configured to permit fluid flow in one direction and prevent fluid flow in an opposite direction, and means 27 for filtering the fluid of a blood vessel, wherein the blood vessel is a superior vena cava or an inferior vena cava. In one embodiment, the filtering means 27 for filtering the fluid of the blood vessel comprises a filter member mounted at an upstream side of the stent member 10. By way of example, a filter member is attached at a proper attaching point on the anchoring member, for example at the attaching points 25A, 25B, 25C, 25D, and 25E on the anchoring members 22A, 22B, 22C, 22D, and 22E, respectively. Other types of venous filtering means are also applicable, for example, stainless steel Greenfield filters by Boston Scientific Corporation (Natick, Mass.), bird's nest filters by Cook, Inc. (Bloomington, Ind.), LGM Vena-Tech filters by B. Braun (Evanston, Ill.), and Simon nitinol filters by Medical Technologies (Woburn, Mass.).

The support structure 26 of the elongate valve stent 21 is configured collapsibly expandable from a first collapsed position to a second expanded position, wherein the stent is delivered through a blood vessel with the support structure in the collapsed position within a delivery apparatus and the stent is secured to a desired valve location at the superior and inferior vena cava with the support structure in the expanded shape and the anchoring means 29 is deployed. In an alternate embodiment, the elongate valve stent 21 with its anchoring means 29 and/or filtering means 27 can be implanted by an open chest procedure at the superior vena cava and the inferior vena cava.

The support structure 26 may be self-expandable, expandable by an inflatable balloon, or by other expanding means. Further, the support structure of the stent member 10 is made of a shape-memory material. One preferred shape-memory material has a first shape transition temperature of between about 30° C. and 45° C. and a second shape transition temperature of between about 25° C. and −20° C., preferably between about 5° C. and −10° C. In operations, the support structure is collapsibly deformed to a small diameter and held at about or below 5° C., preferably between about 5° C. and −10° C. The deformed support structure is then inserted within a delivery apparatus. During a delivery phase, the support structure 26 with its mounted tissue valve 28 is maintained at below the second shape transition temperature by flushing or contacting with super-cooled saline. At a desired location, the elongate valve stent 21 is pushed out of the lumen of the apparatus. Upon reaching the first shape transition temperature, the support structure 26 expands to lock itself in position.

The support structure 26 is made of shape memory Nitinol with at least one shape transition temperature. In one embodiment, the stent or the support structure is sized and configured to be reversibly collapsed by lowering the Nitinol temperature below its second shape transition temperature (for example, about 5° C. and −10° C. in one case) enabling removing the stent or the support structure from a patient percutaneously when needed. This is usually carried out by a retrieval apparatus by grasping the radially deformed device endoluminally.

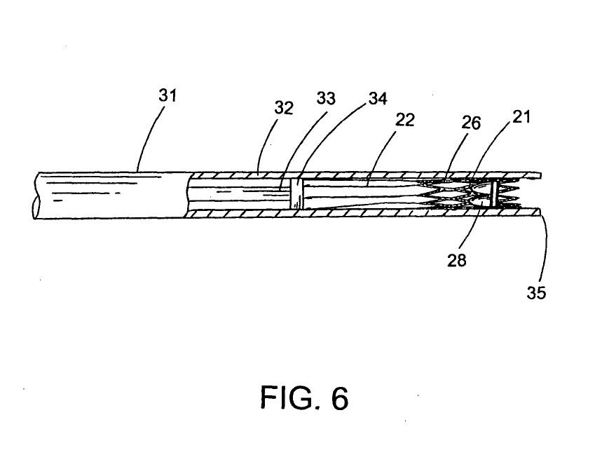

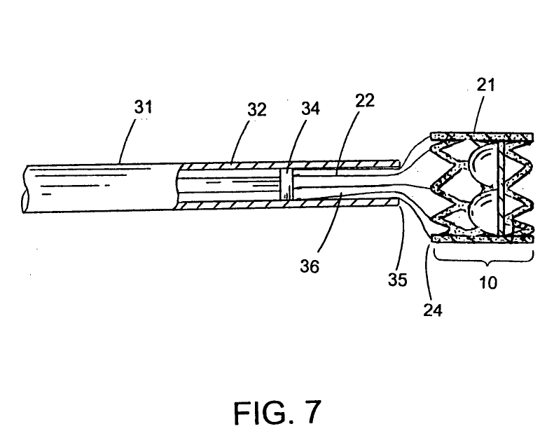

FIG. 6 shows a delivery apparatus 31 with an elongate valve stent 21 at a collapsed position during a delivery phase. In one embodiment, the delivery apparatus 31 is a catheter with a catheter sheath 32 and a lumen 36, wherein a plunger 34 with its pushing rod 33 is used to deploy the valve stent 21 out of the catheter distal end 35. FIG. 7 shows a delivery apparatus 31 with an elongate valve stent 21 at a partially expanded position during a positioning phase. In one embodiment as shown in FIG. 7, the stent member 10 of the valve stent 21 is out of the catheter distal end 35 while a distal hook portion of the anchoring members 22 is still within the lumen 36 of the delivery apparatus 31. When a practitioner continues to advance the plunger 34, the distal hook portion of the anchoring member 22 is deployed out of the catheter distal end 35. When the compressing constraint is removed from the anchoring members 22, the anchoring means 29 tends to recover its resilient preshape and spring outwardly enabling the at least one hook 23 to penetrate and hook into the surrounding tissue.

FIG. 8 shows a preferred embodiment of procedures of protecting a lower body of a patient from high venous pressures, the method comprising implanting an elongate valve stent 21 having a valved stent member 10 suitably placed at an inferior vena cava 56 location, wherein the stent member 10 with a tissue valve 28 is configured to permit blood flow (as indicated by an arrow 58) towards a right atrium 52 of the heart 50 and prevent blood flow in an opposite direction. In a normal patient, the oxygenated blood is pumped from the heart 50 through aorta 51 to the body. Similarly, an elongate valve stent can be implanted at a superior vena cava 55 location for protecting an upper body of a patient from high venous pressure.

Some aspects of the invention relate to a method of protecting an upper or a lower body of a patient from high venous pressures comprising: (a) providing an elongate valve stent, wherein the stent comprises a stent member with a tissue valve secured to a support structure, wherein the support structure is collapsibly expandable, and anchoring means for anchoring the stent member onto surrounding tissue of a vena cava; (b) passing the elongate valve stent through a blood vessel with the support structure in a collapsed position; (c) deploying the stent to an inferior vena cava or a superior vena cava with the support structure in an expanded shape; and (d) securing the stent by anchoring the stent member onto the surrounding tissue of either the superior vena cava or the inferior vena cava with the anchoring means.

The medical device of the invention is for reduction of pressure effects of cardiac tricuspid valve regurgitation. The device does not treat tricuspid valve regurgitation but rather slows down or attempts to block the decay due to the sequels or effects of tricuspid valve regurgitation on the body, namely hepatic dysfunction and renal dysfunction or failure and the build up of fluid in the abdominal cavity and the lower body, legs etc.

Although preferred embodiments of the invention have been described in detail, certain variations and modifications will be apparent to those skilled in the art, including embodiments that do not provide all of the features and benefits described herein. Accordingly, the scope of the present invention is not to be limited by the illustrations or the foregoing descriptions thereof, but rather solely by reference to the appended claims.

1. An elongate valve stent comprising:
a stent member, said stent member comprising a support structure and a tissue valve, wherein said tissue valve is configured to permit fluid flow in one direction and prevent fluid flow in an opposite direction; and
means for anchoring said stent member onto surrounding tissue of a blood vessel.

2. An elongate valve stent comprising:
a stent member, said stent member comprising a support structure and a tissue valve, wherein said tissue valve is configured to permit fluid flow in one direction and prevent fluid flow in an opposite direction; and
means for filtering the fluid of a blood vessel, wherein the blood vessel is a superior vena cava or an inferior vena cava.

3. The elongate valve stent of claim 1, wherein the blood vessel is a vein.

4. The elongate valve stent of claim 3, wherein the blood vessel is a superior vena cava or an inferior vena cava.

5. The elongate valve stent of claim 1 or 2, wherein the support structure is collapsibly expandable from a first collapsed position to a second expanded position.

6. The elongate valve stent of claim 1 or 2, wherein said support structure of the elongate valve stent is self-expandable.

7. The elongate valve stent of claim 1 or 2, wherein said support structure of the elongate valve stent is expandable by an inflatable balloon.

8. The elongate valve stent of claim 1 or 2, wherein the support structure of said stent is made of a shape-memory material having a first shape transition temperature of between about 30° C. and 45° C. and a second shape transition temperature of about 5° C. and −10° C., said support structure being collapsibly deformed to below the second shape transition temperature during a stent delivery phase and expanded after delivery in place upon reaching the first shape transition temperature.

9. The elongate valve stent of claim 1 or 2, wherein said tissue valve has at least one valve leaflet.

10. The elongate valve stent of claim 9, wherein said leaflet is made from a pericardium.

11. The elongate valve stent of claim 10, wherein the pericardium is selected from a group consisting of bovine pericardia, equine pericardia, porcine pericardia, and ovine pericardia.

12. The elongate valve stent of claim 9, wherein said leaflet is chemically treated by a chemical treating agent selected from a group consisting of glutaraldehyde, formaldehyde, dialdehyde starch, epoxy compounds, genipin, and mixture thereof.

13. The elongate valve stent of claim 1 or 2, wherein said tissue valve is a venous valve procured from a group consisting of a bovine jugular vein, an equine jugular vein, a porcine jugular vein, and an ovine jugular vein.

14. The elongate valve stent of claim 1 or 2, wherein said tissue valve is a porcine valve.

15. The elongate valve stent of claim 1 or 2, wherein said support structure is made of a material selected from a group consisting of stainless steel, Nitinol, and plastics.

16. The elongate valve stent of claim 1 or 2, wherein said support structure is coated with a therapeutic agent.

17. The elongate valve stent of claim 16, wherein said therapeutic agent is selected from a group consisting of anticoagulants, antithrombogenic agents, anti-proliferative agents, anti-inflammatory agents, antibiotics, stem cells, growth factors, angiogenesis agents, anti-angiogenesis agents, and statins.

18. The elongate valve stent of claim 1, wherein said anchoring means comprises at least a hook configured for anchoring said stent member onto surrounding tissue of either a superior vena cava or an inferior vena cava.

19. The elongate valve stent of claim 2, wherein said filtering means for filtering the fluid of the blood vessel comprises a filter member mounted at an upstream side of said stent member.

20. A method of protecting an upper or a lower body of a patient from high venous pressures comprising:
providing an elongate valve stent, wherein said stent comprises a stent member with a tissue valve secured to a support structure, wherein the support structure is collapsibly expandable, and anchoring means for anchoring said stent member onto surrounding tissue of a vena cava;
passing the elongate valve stent through a blood vessel with the support structure in a collapsed position;
deploying the stent to an inferior vena cava or a superior vena cava with the support structure in an expanded shape; and
securing the stent by anchoring said stent member onto the surrounding tissue of either the superior vena cava or the inferior vena cava with said anchoring means.

21. The method of claim 20, wherein said tissue valve is configured to permit blood flow towards a right atrium of the patient and prevent blood flow in an opposite direction.

22. The method of claim 20, wherein the step of passing the elongate valve stent endoluminally is through an incision at a blood vessel selected from a group consisting of a jugular vein, a femoral vein, and a subclavian vein.

23. The method of claim 20, wherein the support structure is self-expandable.

24. The method of claim 20, wherein the support structure is expandable by an inflatable balloon.

25. The method of claim 20, wherein the support structure is made of a shape-memory material having a first shape transition temperature of between about 30° C. and 45° C. and a second shape transition temperature of about 5° C. and −10° C., said support structure being collapsibly deformed to below the second shape transition temperature during delivery and expanded after delivery in place upon reaching the first shape transition temperature.

26. The method of claim 20, wherein the support structure is made of a material selected from a group consisting of stainless steel, Nitinol, and plastics.

27. The method of claim 20, wherein the support structure is coated with a therapeutic agent.

28. The method of claim 27, wherein said therapeutic agent is selected from a group consisting of anticoagulants, antithrombogenic agents, anti-proliferative agents, anti-inflammatory agents, antibiotics, stem cells, growth factors, angiogenesis agents, anti-angiogenesis agents, and statins.

29. The method of claim 20, wherein the tissue valve has at least one valve leaflet.

30. The method of claim 29, wherein said leaflet is made from a pericardium.

31. The method of claim 30, wherein the pericardium is selected from a group consisting of a bovine pericardium, an equine pericardium, a porcine pericardium, and an ovine pericardium.

32. The method of claim 20, wherein the tissue valve is chemically treated with a chemical selected from a group consisting of glutaraldehyde, formaldehyde, dialdehyde starch, epoxy compounds, genipin, and mixture thereof.

33. The method of claim 20, wherein the tissue valve is a venous valve procured from a group consisting of a bovine jugular vein, an equine jugular vein, a porcine jugular vein, and an ovine jugular vein.

34. The method of claim 20, wherein the tissue valve is a porcine valve.

35. The method of claim 20, wherein said support structure further comprises filtering means for filtering fluid of the vena cava.
